(19) United States
(12) Patent Application Publication
(10) Pub. No.: US 2006/0229699 A1
(43) Pub. Date: Oct. 12, 2006

(54) Stent-stabilizing device

(76) Inventor: Nasser S. Tehrani, Rome, GA (US)

Correspondence Address: WOOD AND EISENBERG, PLLC, 2121 Eisenhower Ave, Suite 200, Alexandria, VA, 22314, US

(21) Appl. No.: 11/103,431

(22) Filed: Apr. 12, 2005

(51) Int. Cl. A61F 2/06

(52) U.S. Cl. 623/1.11

(57) ABSTRACT

A stent-stabilizing device for controllably reducing the relative motion of a guide wire inside a passageway or lumen of a blood vessel catheter thereby restricting the relative motion of a stent or other object fitted thereto. The stent-stabilizing device comprises an elongated sheath, an expandable plug, and an elongated member. The sheath is preferably tubular in shape. The elongated member comprises an optional handle or grip. In another embodiment the elongated member and sheath are configured to provide a keyway system to prevent unwanted deployment of the plug.

### CROSS-REFERENCE TO RELATED APPLICATIONS

Not Applicable.

### STATEMENT REGARDING FEDERALLY SPONSORED RESEARCH OR DEVELOPMENT

Not Applicable.

### FIELD OF THE INVENTION

This invention relates to a device for controllably reducing the relative motion of a guide wire inside a blood vessel catheter, and more particularly the relative motion of a stent or other object fitted to the guide wire with respect to a target lesion in a blood vessel of a patient.

### BACKGROUND OF THE INVENTION

This invention relates to a device for controllably reducing the relative motion of a guide wire inside a blood vessel catheter and thereby restricting the relative motion of a stent or other object fitted to the distal end of the guide wire with respect to a target lesion in a blood vessel of a patient. Specifically, the invention is a device that is fitted to a stent delivery catheter system for stabilizing a stent while positioning the stent inside a lesion such as a narrowing in a coronary artery. More specifically, the invention is a device that reduces the oscillation or movement of a stent for short periods of time as required by a heart surgeon.

A stent delivery device is typically used by a heart surgeon to deploy and insert a stent or stent-balloon combination about half way along a narrowed section in a patient's coronary blood vessel. If the surgeon is uncertain about the exact dimensions of the narrowed section, such as the length of the narrowed section, it becomes more important to position the stent very carefully inside the narrowed section. Likewise, where a vessel lesion is proximate to a branch in a blood vessel it is very important to position the stent very carefully inside the correct branch of the blood vessel. Heart oscillations can interfere with the operation to correctly insert a stent. The present invention is designed to dampen the movement of a stent during installation or just prior to actual deployment of the stent inside or proximate to a lesion requiring treatment.

While there are numerous published references that describe various types of stent, the applicant is unaware of a teaching or suggestion of an apparatus designed to reduce oscillation of a stent located in a coronary blood vessel. A review of the prior art follows.

U.S. Pat. No. 5,772,669 issued Jun. 30, 1998 to Vrba, describes a stent delivery system that comprises a catheter having a stent receiving portion adapted to receive a stent near the distal end of the catheter and a stent concentrically arranged around the catheter within the stent receiving portion. According to the '669 patent, the stent delivery system further comprises a proximal outer sheath, a retractable distal sheath surrounding at least a portion of the stent and containing the stent in its reduced delivery configuration and a pull back means connected to the retractable distal sheath. The '669 patent also states that the '669 system further comprises an arrangement wherein the retractable sheath is pulled into the catheter when the pull back means is pulled proximally and the distal sheath is retracted, freeing the stent for delivery.

U.S. Pat. No. 5,879,370 issued Mar. 9, 1999 to Fischell et al, describes an expandable stent for use in an artery or other vessel of a human body. According to the '370 patent, the '370 stent structure maintains the patency of the vessel within which the stent is expanded radially outward. In one claimed embodiment of the '370 device a stent having a multiplicity of frames joined together by at least two undulating longitudinal structures that can readily change their length in the longitudinal direction to provide increased longitudinal flexibility for the stent for easy passage through and placement within a curved vessel such as a coronary artery.

U.S. Pat. No. 6,254,611 issued Jul. 3, 2001 to Vrba, describes a stent delivery system that comprises a catheter with a retractable sheath arranged concentrically near its distal end. According to the '611 patent, the retractable sheath is connected to a pull wire for retraction. A sliding seal connects the proximal end of the retractable sheath and the catheter to form a fluid tight chamber when the retractable sheath is in the unretracted position. The '611 device is prepared by filling the fluid tight chamber with fluid to flush out air prior to insertion into the vasculature of the patient.

U.S. Pat. No. 6,565,601 issued May 20, 2003 to Wallace et al, describes a method for vascular reconstruction of diseased, non-aneurysmal arteries. According to the '601 patent, the method comprises the steps of: (a) identifying the vascular site of a diseased, non-aneurysmal artery in a mammalian patient wherein the vascular site participates in the systemic blood flow of the patient; (b) inserting a stent into the diseased artery at the vascular site; and (c) delivering a fluidic composition to the vascular site which composition in situ forms a solid in and around the stent thereby isolating the vascular walls at the vascular site from systemic blood flow while retaining blood flow through the artery.

U.S. Pat. No. 6,572,646 issued Jun. 3, 2003 to Boylan et al and U.S. Publication Number 20030187497 published Oct. 2, 2003 to Boylan et al, describe a curved nitinol stent for extremely tortuous anatomy. The '646 device is used in a curved body lumen. The '646 stent is said to be made from a super-elastic alloy such as nickel titanium or nitinol, and optionally includes a ternary element. The super-elastic alloy has a low temperature phase or martensitic phase and a high temperature phase or an austenitic phase. In the high temperature phase, the stent has a curve along the length that closely matches the curve of the vessel in the patient's anatomy. When deployed in the curved vessel of the patient, the heat set curve of the stent closely conforms to the curvature in the vessel and minimizes trauma and stress to the vessel.

U.S. Pat. No. 6,579,297 issued Jun. 17, 2003 to Bicek et al, describes a stent delivery system that includes a catheter having a retractable outer sheath near its distal end. A shape memory contraction member having a memorized contracted shape is connected to the retractable outer sheath. A heat generating device connected to the shape memory contraction member causes the shape memory contraction member to heat up to its transition temperature and assume its contracted position, retracting the retractable outer sheath. Another embodiment utilizes 2 springs, a “normal” spring and a shape memory alloy (SMA) spring, the two springs selected and designed so that the “normal” has an expansion force which is less than SMA spring when the SMA spring is austenitic, but greater than the SMA spring when the SMA spring is martensitic. Yet another embodiment utilizes a shape memory latch that in its martensitic state abuts a stop to prevent a spring from moving the sheath proximally, but in its austenitic state releases the stop, allowing the spring to retract the sheath to release the stent for deployment.

U.S. Publication Number 20010027323 published Oct. 4, 2001 to Sullivan et al, describes a stent delivery system for delivering a self-expanding stent to a predetermined location in a vessel. The '323 stent includes a catheter body having an axial guide-wire lumen and a pull-wire lumen. A medical device such as a self-expanding stent is held in a reduced delivery configuration for insertion and transport through a body lumen to a predetermined site for deployment. The stent is carried axially around the catheter body near its distal end and held in its reduced configuration by a retractable outer sheath. A proximal retraction handle is connected to the proximal end of the catheter body and includes a pistol grip trigger engaging a ratchet mechanism, which is connected to a pull-wire that extends through the pull-wire lumen and is connected to the retractable outer sheath.

U.S. Publication Number 20020143381 published Oct. 3, 2002 to Gilligan et al, describes an intraluminal prosthesis composed of a self-expandable stent and a biodegradable constraining element being capable of biodegrading in vivo over a predetermined period of time to permit radial expansion of the stent. The constraining elements are applied to the stent to produce a compressed configuration. Dissolution of the constraining elements in vivo allows for expansion of the stent to an expanded configuration.

U.S. Publication Number 20020169496 published Nov. 14, 2002 to Wallace et al, describes a method for treating a diseased, non-aneurysmal artery in a mammalian patient. According to the '496 publication, the method comprises the steps of: (a) identifying the vascular site of a diseased, non-aneurysmal artery in a mammalian patient wherein said vascular site participates in the systemic blood flow of said patient; (b) inserting a stent into the diseased artery at the vascular site; and (c) delivering a fluidic composition to the vascular site which composition in situ forms a solid in and around the stent thereby isolating the vascular walls at the vascular site from systemic blood flow.

W.I.P.O. International Application Number WO-99-49808 published Oct. 7, 1999 to Gilson et al, describes a rapid exchange stent delivery catheter for delivery and deployment of a stent. The '808 stent has a catheter shaft having a guidewire lumen defined by a passageway with an entrance and an exit. The '808 stent is made of a shape memory metallic alloy and is constrained by a sheath. The sheath has an elongate slot aligned with the guidewire lumen entrance so that a guidewire is not obstructed during movement of the sheath to deploy the stent. The sheath is pulled back linearly by a thumbscrew mechanism to deploy the stent.

DYNALINK™ have a self-expanding stent system with a 0.035″ guide wire. The DYNALINK™ stent system is said to provide 6F sheath/8F guiding catheter compatibility for 5.0-10.0 mm diameters in five stent lengths (28, 38, 56, 80 and 100 mm) and two catheter lengths (80 and 120 cm). The 12 and 14 mm diameters are 7F sheath/9F guiding catheter compatible and come in three stent lengths (38, 56 and 80 mm) and three catheter lengths (55, 80 and 120 cm).

None of the above patents and publications, taken either singly or in combination, is seen to describe the instant invention as claimed.

### SUMMARY OF THE INVENTION

The present invention is directed to a stent-stabilizing device for controllably reducing the relative motion of a guide wire inside a passageway or lumen of a blood vessel catheter thereby restricting the relative motion of a stent or other object fitted thereto. The stent-stabilizing device comprises an elongated sheath, an expandable plug, and an elongated member. The sheath is preferably tubular in shape. The elongated member comprises an optional handle or grip. In another embodiment the elongated member and sheath are configured to provide a keyway system to prevent unwanted deployment of the plug.

Accordingly, it is a principal object of the invention to provide a stent-stabilizing device for controllably reducing the relative motion of a guide wire inside a passageway or lumen of a blood vessel catheter thereby restricting the relative motion of a stent or other object fitted thereto.

This and other objects of the present invention will become readily apparent upon further review of the following specification and drawings.

### BRIEF DESCRIPTION OF THE DRAWINGS

FIG. 1A is a partial perspective environmental view of a stent-stabilizing device inserted into a conventional angioplasty apparatus according to the present invention.

FIG. 1B shows the stent-stabilizing device of FIG. 1A with an expandable plug member in deployed configuration pressed against a section of guide wire.

FIG. 1C is a perspective environmental view of a stent-stabilizing device of FIG. 1 but fitted with a grip.

FIG. 2A shows a first embodiment of the stent-stabilizing device according to the present invention.

FIG. 2B shows the stent-stabilizing device of FIG. 2A with an expandable plug member in deployed configuration according to the present invention.

FIG. 2C shows a further embodiment of the stent-stabilizing device according to the present invention.

FIG. 2D shows the stent-stabilizing device of FIG. 2C with the expandable plug member in deployed configuration according to the present invention.

FIG. 2E shows a further embodiment of the stent-stabilizing device according to the present invention.

FIG. 3A shows another embodiment of the stent-stabilizing device according to the present invention, wherein the plug member takes the form of an expandable mesh.

FIG. 3B shows the stent-stabilizing device of FIG. 3A with the expandable plug member in deployed configuration according to the present invention.

FIG. 3C shows another embodiment of the stent-stabilizing device according to the present invention.

FIG. 4 shows another embodiment of the stent-stabilizing device according to the present invention.

FIG. 5A shows yet another embodiment of the stent-stabilizing device according to the present invention incorporating an anti-plug deployment mechanism.

FIG. 5B shows a close up view of the anti-plug deployment mechanism of FIG. 5A.

Similar reference characters denote corresponding features consistently throughout the attached drawings. It will be understood that the terms “FIG.” and “Figure” are regarded as equivalent terms.

### DETAILED DESCRIPTION OF THE PREFERRED EMBODIMENTS

The present invention is a stent-stabilizing device for controllably reducing the relative motion of a guide wire inside a passageway or lumen of a blood vessel catheter thereby restricting the relative motion of a stent or other object fitted thereto. The stent-stabilizing device of the present invention is particularly suitable for steadying a stent at the site of a target lesion in a blood vessel of a patient.

It should be understood that the term “stent” as used herein is also intended to cover any object or combination of objects that can be attached, for example, to the distal end of a catheter wire. For example, the term “stent” covers stent-balloon combinations that are employed, for example, in coronary blood vessel surgery.

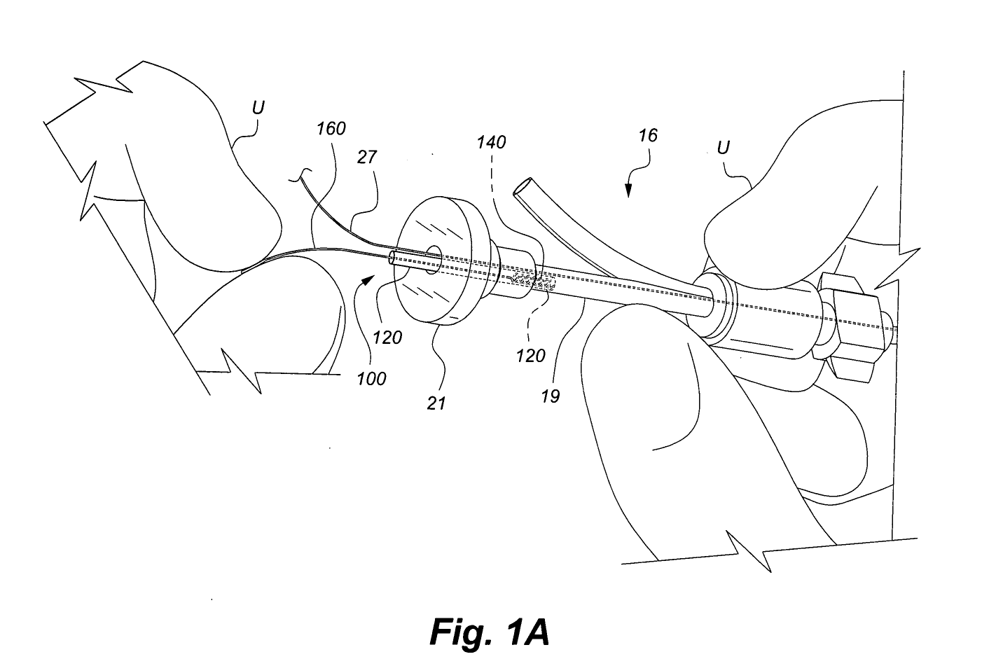

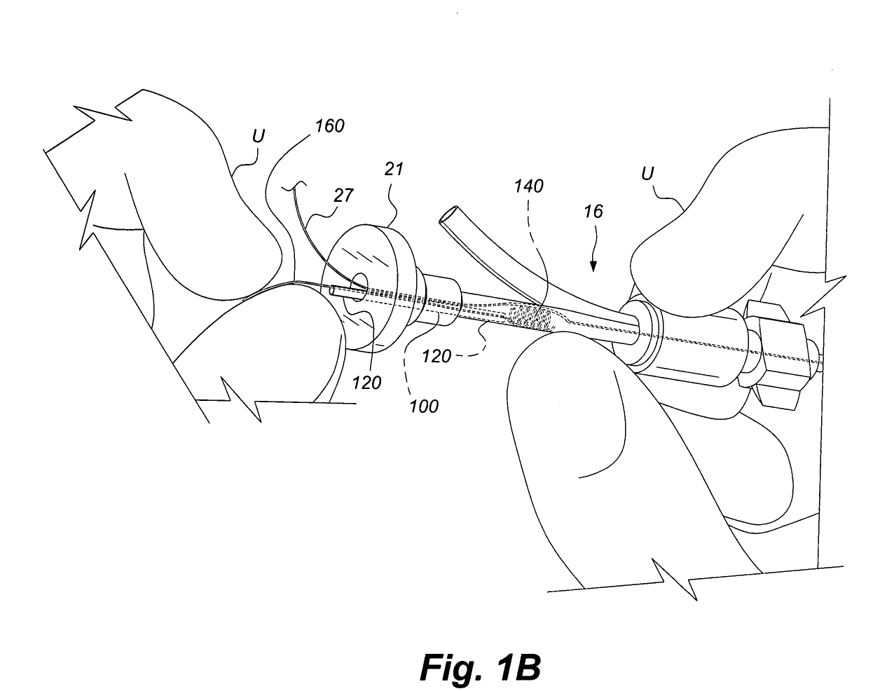

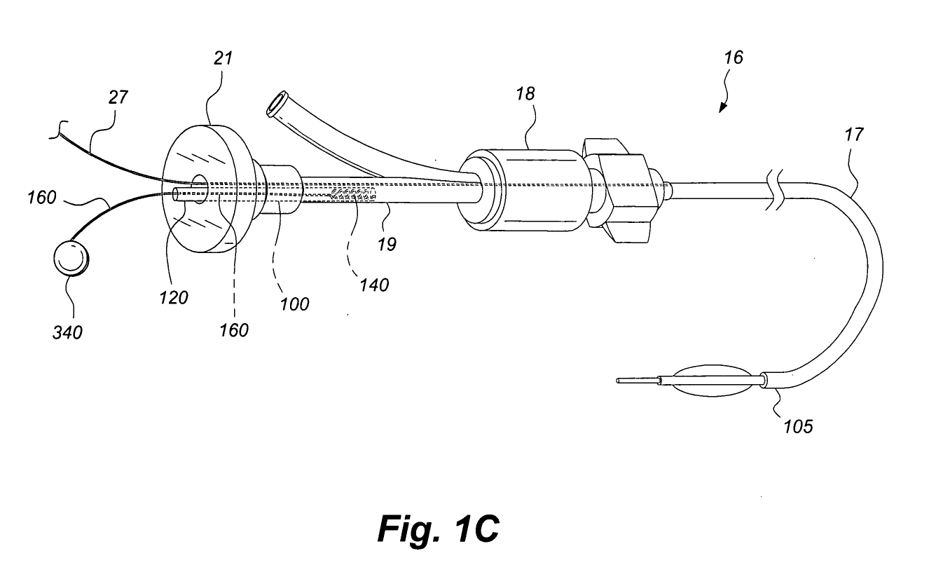

Referring to the FIGURES in general, the stent-stabilizing device of the invention is denoted by the reference numeral 100 as a whole. The stent-stabilizing device 100 comprises an elongated sheath 120, an expandable plug 140, and an elongated member 160. The sheath 120 is preferably tubular in shape. The elongated member 160 comprises an optional handle or grip 340. It should be understood that the length of member 160 can be any suitable length; e.g., the length of elongated member 160 could be sufficient to permit the plug 140 to deploy midway along and inside the lumen of the guiding catheter 17 shown in FIG. 1C; alternatively, the length of member 160 could be sufficient to permit the deployment of plug 140 at the distal end 105 of catheter 17.

Referring to the FIGURES in general and FIGS. 1A, 1B, and 1C in particular; FIGS. 1A, 1B are environmental perspective views of stent-stabilizing device 100 inserted into a conventional angioplasty apparatus 16 (as disclosed, for example, in U.S. Pat. No. 6,575,993); FIG. 1C is a side elevational view of the angioplasty apparatus 16 incorporating the present invention 100. In more detail, device 100 is shown inserted into knurled knob 21 at the proximal end of the conventional angioplasty apparatus 16, which comprises a conventional guiding catheter 17, a guide wire 27 extending through the y-adapter 19 and guiding catheter 17.

In FIGS. 1A, 1B and 1C device 100 is inserted alongside guide wire 27. However, it is not essential that the device 100 be inserted alongside the guide wire 27. As described below, the device 100 may be inserted into a guide wire passageway or lumen at a different point from the actual insertion point for the guide wire 27. The device can be inserted via knurled knob 21 and the guide wire 27 via, for example, a notch. In short, the entry point for the device 100 is not critical provided the expandable plug 140 is deployable alongside a section of the guide wire 27.

It should be understood that the device 100 is not limited to a particular type of medical catheter and can be used with a variety of medical catheters that employ a guide wire. For example, device 100 can be used with angioplasty apparatus as described in U.S. Pat. No. 5,685,312 (issued Nov. 11, 1997 to Yock) and U.S. Pat. No. 6,575,993 (issued Jun. 10, 2003 to Yock); the Yock '312 and '993 patents are incorporated herein by reference in their entirety.

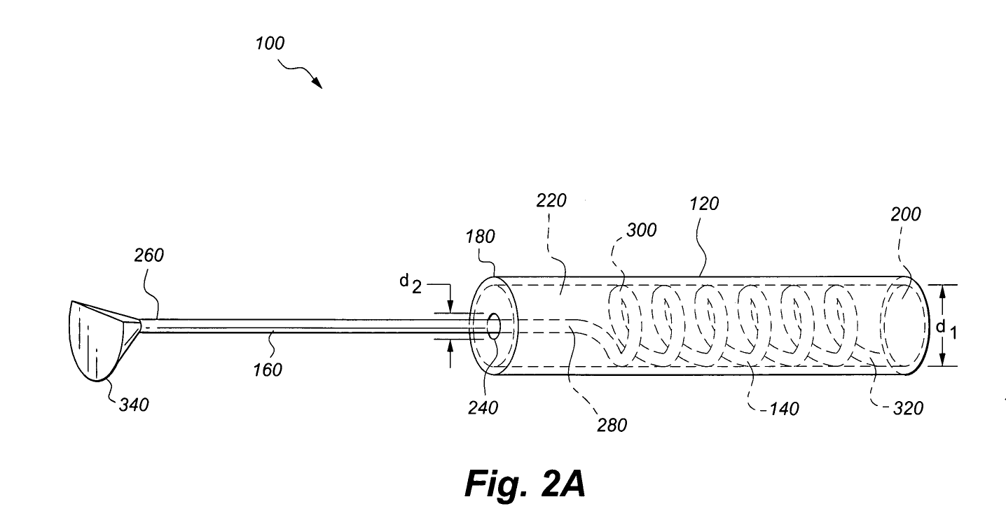

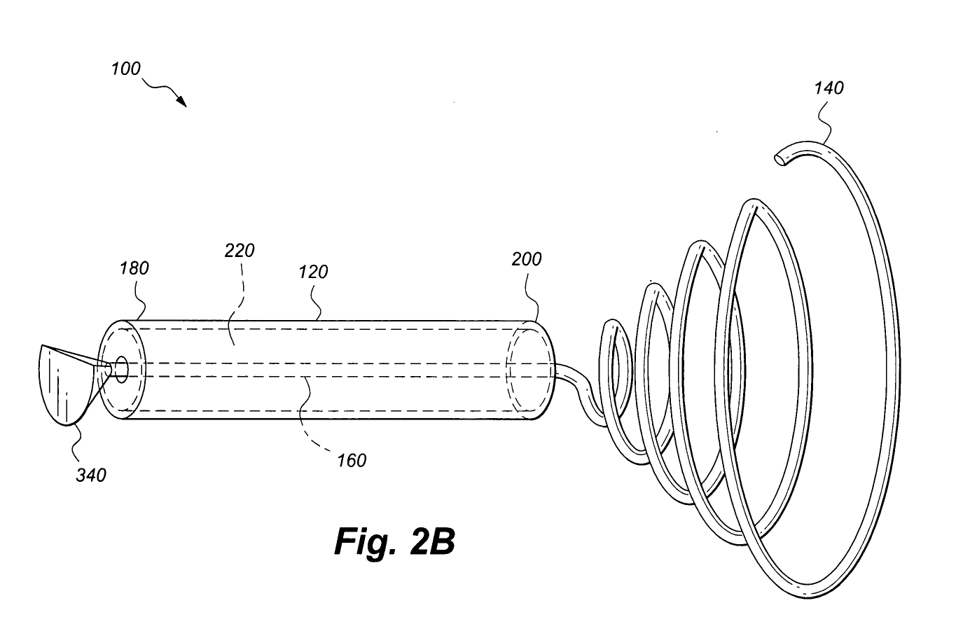

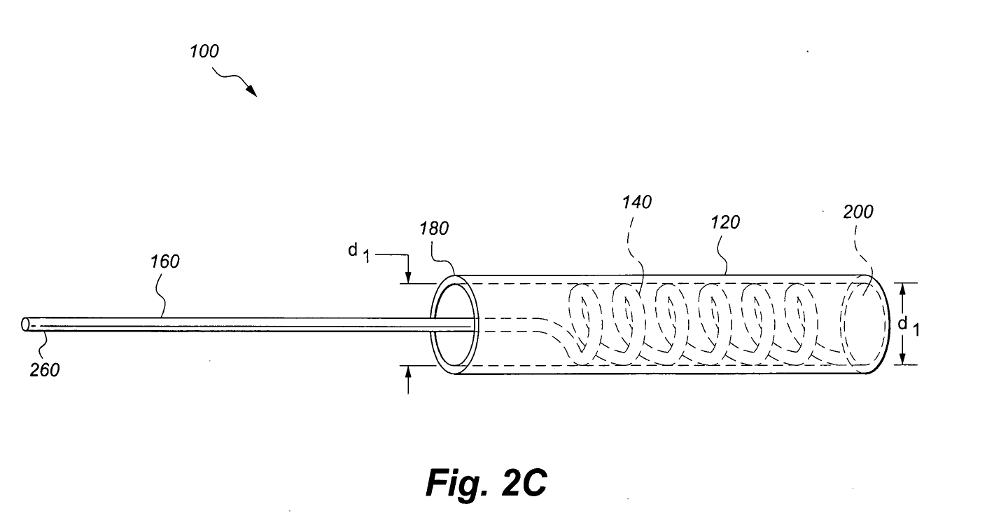

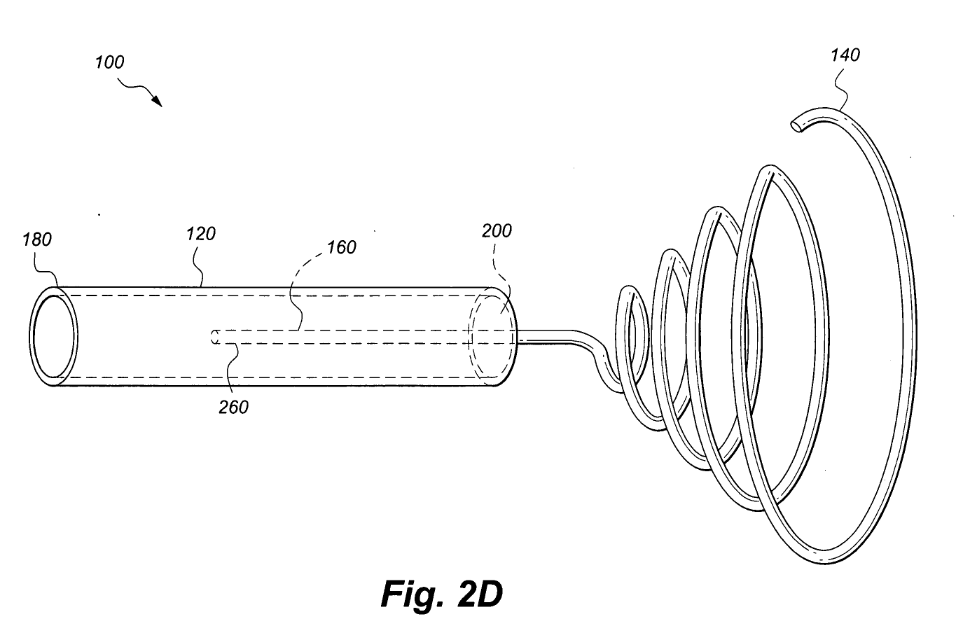

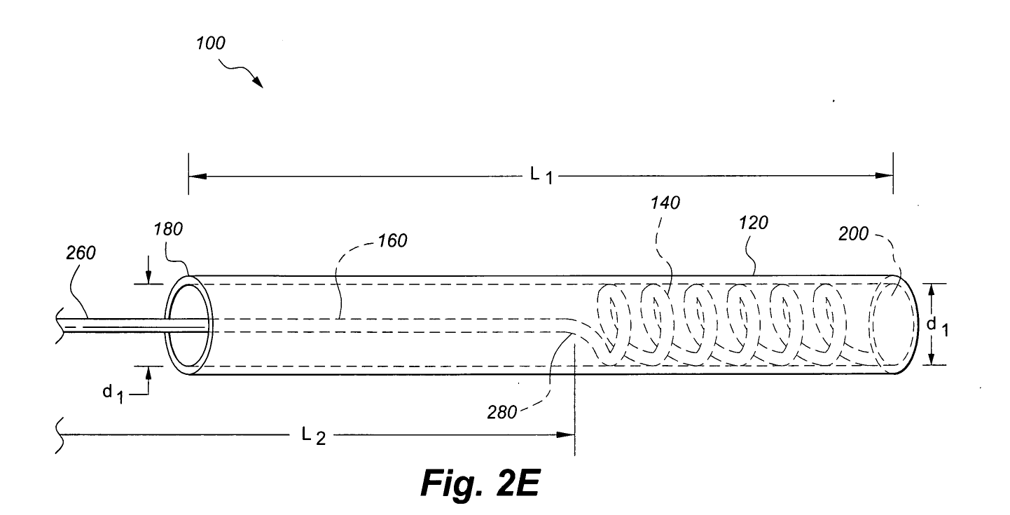

In addition, the device 100 can be used in medical catheters where a guide wire is inserted into a guide wire passageway or lumen via a notch such as the notch described in U.S. Pat. No. 4,748,982 (element number 32 in the '982 patent, which was issued Jun. 7, 1988 to Horzewski et al); the Horzewski et al '982 patent is incorporated herein by reference in its entirety. The device 100 can be inserted into the guidewire passageway at a different access point providing the lengths L1 and L2 (FIG. 2E) of the sheath 120 and elongated member 160, respectively, are sufficient to ensure the plug 140 can be deployed between the notch (element number 32 in the Horzewski et al '982 patent) and the distal end of the '982 apparatus to press the guide wire against the inside of the guidewire passageway or lumen (member number 16 in the '982 patent); alternatively, at the appropriate moment the user U (FIGS. 1A and 1B) can insert or squeeze the device 100 into the notch (element number 32 in the Horzewski et al '982 patent) alongside the guide wire to controllably deploy the expandable plug 140. It should be understood that the term “controllably deploy” refers to the voluntary act of a user U, such as a cardiologist, in deploying the plug 140 outside and downstream of the distal end 200 of sheath 120.

Expandable plug 140 is shown in non-deployed and deployed configurations in FIGS. 1A and 1B, respectively. Specifically, in FIG. 1A expandable plug 140 is in a compressed state inside sheath 120, i.e., sheath 120 serves to restrain plug 140 in its compressed state; and in FIG. 1B the expandable plug 140 is in an expanded state as occurs when deployed outside and downstream of sheath 120. It should be understood that the terms “expanded state”, “expanded shape”, “first predetermined shape”, and “first predetermined state” are regarded as equivalent terms. It should also be understood that the terms “compressed state”, “compressed shape”, “second predetermined shape” and “second predetermined state” are regarded as equivalent terms.

Still referring to FIGS. 1A and 1B, user U is shown holding the distal end of the elongated member 160. User U deploys the expandable plug 140 by pushing the plug 140 out of the sheath 120 by means of the elongated member 160. Alternatively, user U deploys the plug 140 by pulling the sheath 120 towards user U while holding the elongated member 160 stationary. User U can combine both methods simultaneously using the fingers of both hands to pull the sheath 120 while pushing the plug 140 out of the end of the sheath. It should be immediately apparent that the plug 140 is in a compressed state inside the sheath 120 and in an expanded state when deployed from sheath 120.

FIG. 2A shows a first embodiment of device 100 in more detail. Elongated sheath 120 comprises opposite open ends, namely proximal open end 180 and distal open end 200. An internal hollow bore 220 of internal diameter d₁ is disposed between the proximal 180 and distal end 200 of sheath 120; the hollow bore 220 is sized to accommodate the compressed form of expandable plug 140. The proximal open end 180 takes the form of an aperture 240. The diameter d₂ of aperture 240 should be sufficient to accommodate the cross-section area of elongated member 160 while preventing the expandable plug 140 from inadvertently exiting from the proximal end 180 of sheath 120. The internal diameter d₁ of bore 220 (and hence inner diameter of open distal end 200) should be sufficient to allow the compressed form of plug 140 to exit bore 220. The length L1 (see FIG. 2E) of the elongated member 160 should be sufficient to allow a user U to controllably push plug 140 from bore 220 and deploy plug 140 downstream of sheath 120.

Expandable plug 140 exists either in a compressed state inside bore 220 of sheath 120 or in an expanded state deployed outside sheath 120. Expandable plug 140 is slidably movable inside bore 220 of sheath 120. In its compressed state the expandable plug 140 can be moved back and forth along bore 220; specifically, a user U manipulates elongated member 160 to slidably move the plug 140 back and forth inside bore 220 of sheath 120. Alternatively, user U manipulates sheath 120 to alter the position of plug 140 inside bore 220.

Still referring to FIG. 2A, elongated member 160 comprises opposite ends, namely proximal end 260 and distal end 280; and expandable plug 140 comprises proximal end 300 and distal end 320. The distal end 280 of elongated member 160 is attached to the proximal end 300 of the expandable plug 140. The proximal end 260 of elongated member 160 terminates in optional grip 340. The optional grip 340 may either form an integral part of proximal end 260 or be attached to end 260. FIG. 2C shows proximal end 260 of elongated member 160 lacking optional grip 340. The elongated member 160 can be made of any suitable material such as a thin stiff wire between about 0.05″ and 0.2″ in diameter, and more preferably about 0.14″ in diameter.

FIG. 2B shows the stent-stabilizing device 100 of FIG. 1A with expandable plug 140 in a deployed configuration. The elongated member 160 has been pushed into the sheath 120 thereby deploying the expandable plug 140 through distal end 200 of sheath 120. The diameter d₂ (shown in FIG. 2A) of aperture 240 is also chosen to prevent the optional grip 340 from entering the bore 220 of sheath 120; in addition, the combination of aperture 240 and optional grip 340 prevents the proximal end 180 of sheath 120 from sliding over and off proximal end 260 of elongated member 160.

FIG. 2C shows an alternative embodiment of the stent-stabilizing device 100. In this embodiment proximal end 180 has an open end that lacks aperture 240. In this embodiment the sheath 120 can be slid along the elongated member 120 and off proximal end 260. A user U, such as a heart surgeon, may desire to pull the sheath 120 off the proximal end 260 leaving the plug 140 deployed and expanded up against a section of guide wire 27 as shown in FIG. 1B. Specifically, the expanded plug 140 pushes the guide wire 27 up against the interior of the guide wire passageway or lumen (see, e.g., FIG. 1B) thereby reducing any back and forth movement or the propensity for movement in the guide wire 27 and by default any device such as, for example, a stent or stent/balloon combination attached to the distal end of the guide wire 27. A user U may desire reduction in the movement of the guide wire 27 at a critical point in blood vessel surgery; the device 100 provides the user U with the ability to deploy an expandable plug 140 to controllably reduce movement in the guide wire 27 and any object such as a stent attached to the distal end of the guide wire 27.

FIG. 2D shows the stent-stabilizing device 100 of FIG. 2C in which the sheath 120 is pulled over the proximal end 260 thereby deploying plug 140, which expands to its expanded state. Thus, a user U can deploy the plug 140 by dragging the sheath 120 off the proximal end 260 of elongated member 160. The ability to completely remove the sheath 120 can be useful when the user U needs to free his/her fingers from holding the sheath 120.

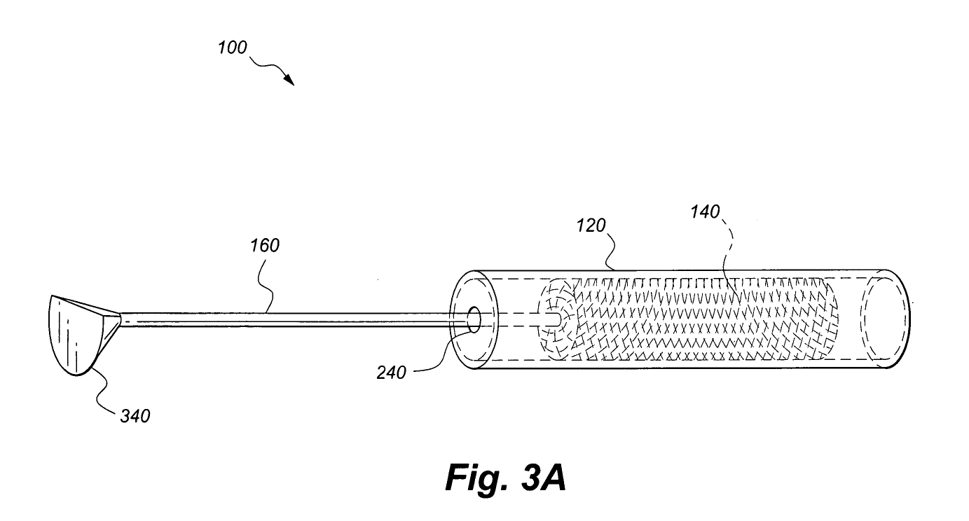

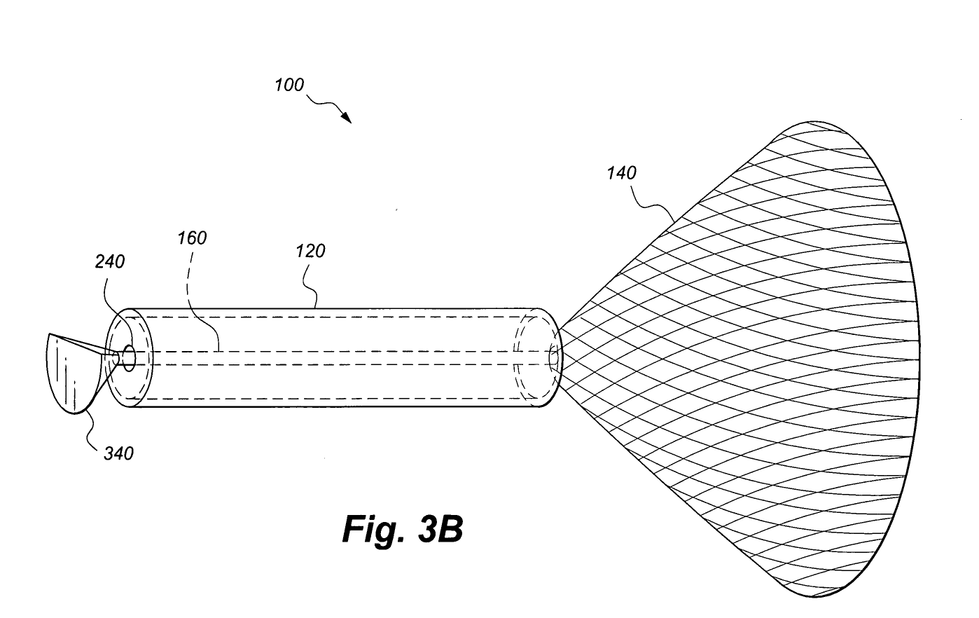

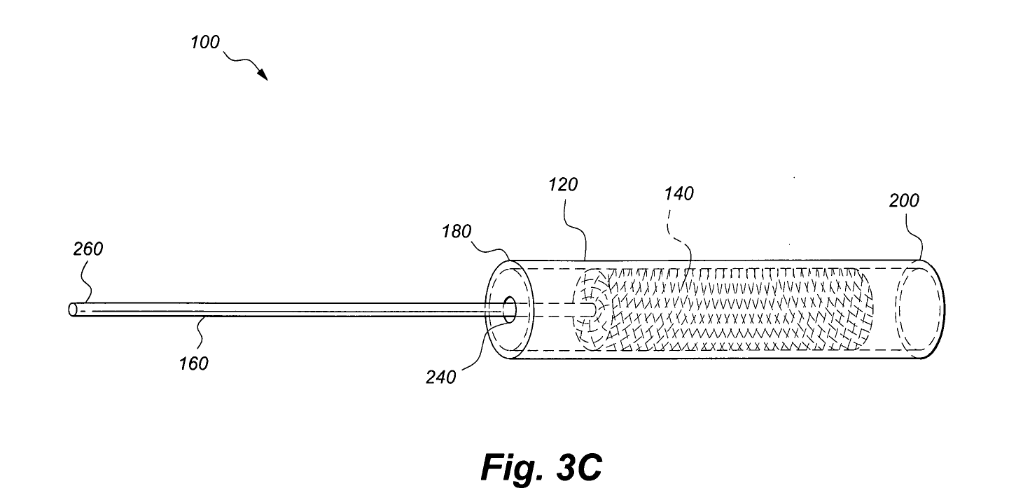

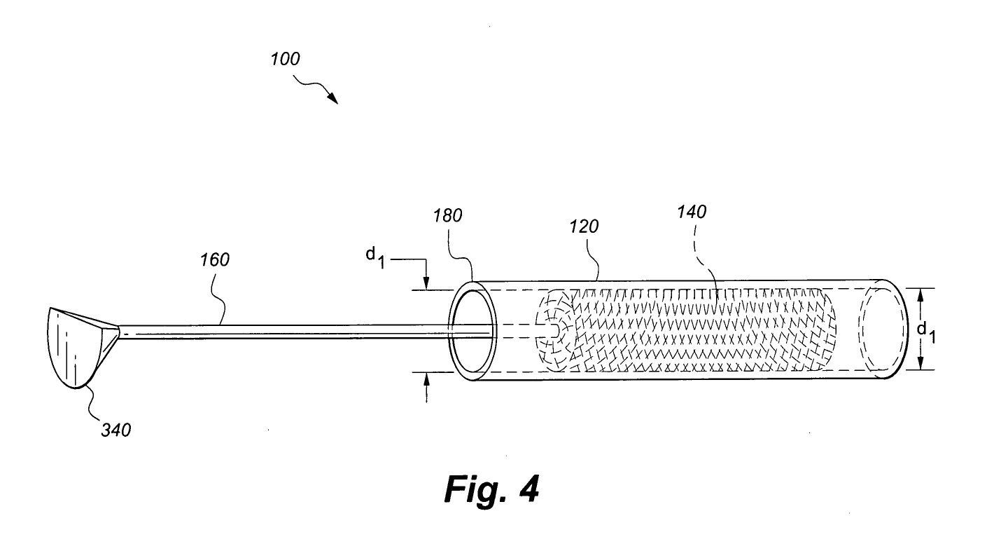

In FIGS. 2A, 2B, 2C and 2D, expandable plug 140 takes the form of an expandable spring which is compressed when stored inside bore 220 and expands to an expanded state when deployed as shown in FIGS. 2B and 2D. In contrast, in FIGS. 3A, 3B, and 3C expandable plug 140 takes the form of an expandable mesh like structure that is compressed when stored inside bore 220 (as shown in FIG. 3A) and expands to an expanded state when deployed as shown in FIG. 3B. FIG. 4 shows grip 340 fitted to elongated member 160 shown in FIG. 3C, and more particularly to proximal end 260.

The shape of the plug 140 is preferably adapted to allow the expanded form of the plug 140 to be pulled back into the bore 220 of sheath 120 thereby allowing repeated reuse of device 100. It is preferred that the proximal end 300 (see FIG. 2A) of the expanded form of plug 140 is tapered to allow easy retraction back into the sheath 120. For example, the plug 140 can take the form of a conical shaped spring such as a conical shaped Nitinol coiled wire spring.

The diameter of the compressed form of plug 140 can vary depending on the internal diameter of the entry point in apparatus 16 that in turn determines the maximum permissible diameter of sheath 120 (and more particularly the maximum permissible diameter of bore 220) that in turn determines the maximum permissible diameter of the compressed form of plug 140. The dimensions of the expanded form of plug 140 are selected to ensure that upon deployment the expanded plug 140 is sufficiently large to ensure a section of the guide wire 27 is held firmly against the interior wall of the passageway such as that of a catheter or lumen housing the guide wire 27.

It should be understood that the expandable plug 140 is not limited to expandable springs or expandable mesh plugs; the expandable plug can take any suitable form and any suitable shape. For example, the expandable plug 140 can have a cylindrical shape. In addition, the expandable plug 140 may be made of any suitable material such as stainless steel. However, it is preferred that the expandable plug is made of smart memory material such as a memory shape alloy. It is preferred that the expandable plug 140 remembers its expanded shape such that if the plug 140 is compressed and later released from compression, the plug 140 reverts back to its memory expanded shape. It is preferred that the plug 140 comprises Nitinol, a nickel-titanium shape memory alloy that acts like a super-elastic material that has shape memory. A plug manufactured out of Nitinol would provide the plug 140 with the ability to expand to its memory-expanded shape. The method of imposing a memory-expanded shape on an object manufactured from Nitinol is known to persons of ordinary skill in the art and is described, for example, in U.S. Pat. No. 5,147,370 issued Sep. 15, 1992 to McNamara et al; the McNamara et al '370 patent is incorporated herein by reference in its entirety.

The shape memory alloy Nitinol is made into a first predetermined shape above a transition temperature range (TTR), the TTR being dependent on the particular ratio of metals in the Nitinol alloy. Below the TTR the alloy is highly ductile and may be plastically deformed into a second desired shape such as a compressed or crumpled state. Above the TTR, and in the absence of physical restraint, the alloy returns to its first pre-set form.

If made of Nitinol alloy, the expandable plug 140 is made by shaping it into a first predetermined shape above a transition temperature range (TTR). At a temperature below the TTR the plug 140 is plastically deformed into a second desired state (i.e. the compressed state of plug 140) that can fit inside bore 220. The sheath 120 serves to restrain the compressed version of plug 140 even when the surrounding temperature is above the TTR. Upon deployment outside and downstream of sheath 120, the Nitinol plug 140 expands to the first predetermined shape, i.e. the expanded state; it will be understood that the ratio of Ni/Ti (i.e., Nickel/Titanium) is chosen such that the ambient temperature found in an operating room is above the TTR of the Nitinol plug 140; for example a TTR between about 35° F. and 75° F., and more preferably between about 40° F. and 50° F. The sheath 120 serves to physically restrain the Nitinol plug 140 in its deformed or compressed state at temperatures above the TTR, but upon deployment outside of sheath 120, and hence free of the physical restraint of the walls of sheath 120, the plug 140 immediately expands from its compressed state to its first pre-set form such as shown, for example, in FIG. 1B.

Plug 140 can be made out of Nitinol coil, i.e. solid nitinol wire. For example, the expandable plug 140 can be constructed from a nickel/titanium metal alloy (Nitinol) wire in the amount of about 55% nickel and 45% titanium. The diameter of the wire can vary with a preferred diameter between about 0.001 to about 0.038 inch. The use of nitinol in medical devices is well known in the art. Nitinol is preferred because of its super-elasticity and its shape memory. However, other solid materials that are also elastic or super-elastic and have shape memory could also be used such as some synthetic plastics, metallic alloys, and the like. To make the Nitinol coil form of plug 140, nitinol wire is wrapped around a suitably shaped mandrel to form a Nitinol wire coil configuration. The nitinol coil is then heated to an appropriate temperature such that the nitinol wire adopts the coil configuration as its resting shape upon cooling. Because nitinol is super-elastic, the nitinol version of plug 140 is compressed with the use of a small amount of force to fit inside sheath 120 and then reform to its natural resting configuration upon exiting the distal end 200 of sheath 120.

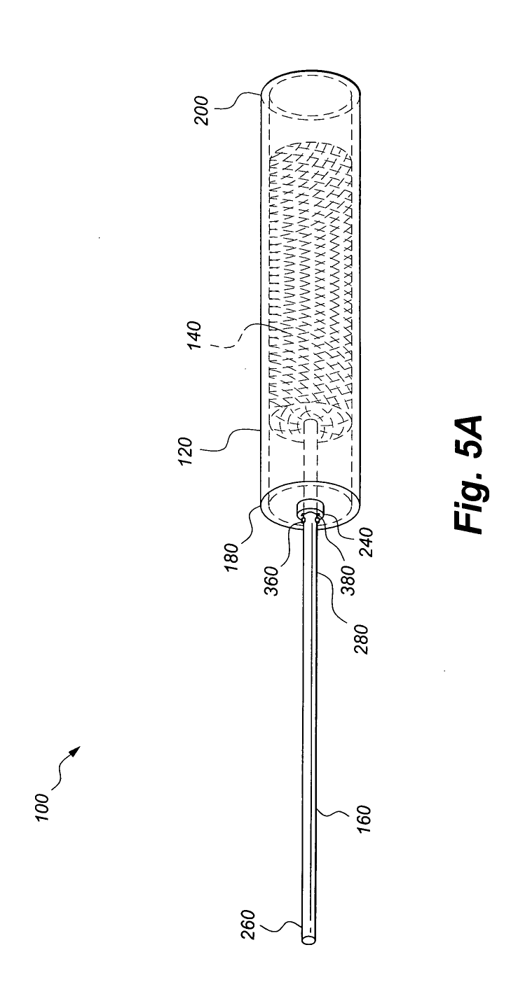

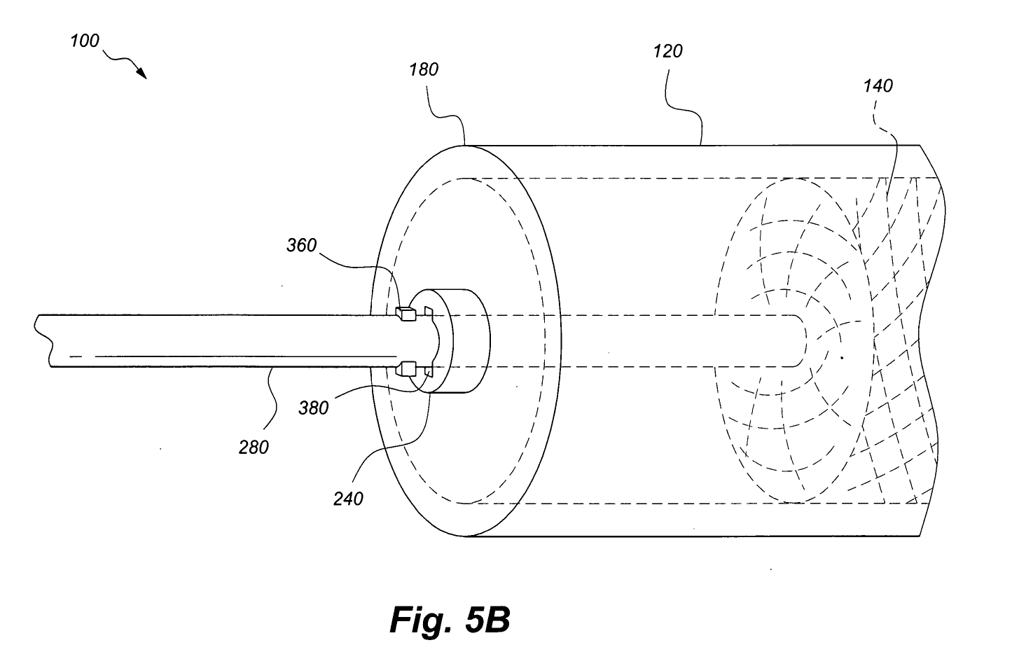

FIGS. 5A and 5B show a further embodiment of device 100 in which the device 100 is includes an anti-plug deployment mechanism to prevent unwanted or otherwise inadvertent or accidental deployment of plug 140. Specifically, the elongated member 160 and aperture 240 are modified to prevent the plug 140 accidentally or inadvertently deploying out of distal end 200. As can be seen in FIGS. 5A and 5B the distal end 280 of elongated member 160 comprises at least one protrusion 360, and aperture 240 comprises at least one complementary slit or groove 380, wherein the protrusion(s) 360 and groove(s) 380 form a keyway style setup to prevent accidental deployment of the plug 140 (e.g., to prevent accidental deployment of the plug 140 during transit and/or packaging of stent stabilizing device 100). Specifically, such an arrangement helps prevent accidental deployment of the expandable plug 140 from distal end 200 thereby preventing the plug 140 from expanding at an inopportune time or otherwise inappropriate time during, for example, heart surgery.

Still referring to FIGS. 5A and 5B, to deploy the plug 140 a heart surgeon would simply twist the elongated member 160 until the protrusions 360 align with slits or grooves 380 and then push the elongated member 160 out of the sheath 120 to deploy plug 140. Thus, the protrusion(s) 380 and slits or grooves 380 are complementary, i.e., designed to work cooperatively to prevent unwanted deployment of the plug 140.

It would be understood by persons of ordinary skill in the art that other anti-plug deployment mechanisms can be built into the device 100 and the example shown in FIGS. 5A and 5B is not intended to limit the present invention to just one type of anti-plug deployment mechanism.

It is to be understood that the present invention is not limited to the embodiments described above, but encompasses any and all embodiments within the scope of the following claims.

I claim:

1. A stent-stabilizing device for pressing a section of guide wire against the interior wall of a guidewire passageway to reduce the relative motion of a stent or other object attached to the distal end of the guide wire, comprising:
an elongated sheath having a hollow bore, the sheath having open proximal and distal opposite ends, wherein the sheath can fit inside the bore of a guide wire passageway;
an expandable plug having proximal and distal ends, wherein the plug exists either in a compressed state inside the sheath or in an expanded state deployed outside the sheath; and
an elongated member having proximal and distal opposite ends, wherein the distal end of the elongated member is attached to the proximal end of the expandable plug,
whereby the expandable plug is slidably movable inside the hollow bore and upon deployment outside the sheath expands to a predetermined shape to press up against a section of guide wire to reduce the motion of the guide wire and an object attached to the distal end of the guide wire.

2. The stent-stabilizing device of claim 1, wherein the expandable plug comprises a smart shape memory alloy, further wherein the plug adopts a predetermined first shape and upon compression adopts a predetermined second shape.

3. The stent-stabilizing device of claim 1, wherein the expandable plug is a conical shaped Nitinol coiled wire spring that adopts a predetermined first shape and upon compression adopts a predetermined second shape.

4. The stent-stabilizing device of claim 1, wherein the elongated member is a thin stiff wire between about 0.05″ and 0.2″ in diameter.

5. The stent-stabilizing device of claim 1, wherein the elongated member is a thin stiff wire having a diameter of about 0.14″.

6. The stent-stabilizing device of claim 1, wherein the expandable plug comprises Nitinol in the amount of about 55% nickel and 45% titanium.

7. The stent-stabilizing device of claim 1 further comprising a grip attached to the proximal end of the elongated member.

8. The stent-stabilizing device of claim 1, wherein the proximal open end of the sheath defines an aperture of sufficient diameter to accommodate the cross-section area of the elongated member while preventing the expandable plug from inadvertently exiting from the proximal end of the sheath.

9. The stent-stabilizing device of claim 8, wherein the aperture and elongated member collectively define a keyway to prevent accidental deployment of the plug.

10. The stent-stabilizing device of claim 8, wherein the distal end of the elongated member comprises at least one protrusion and the aperture defines at least one complementary slit to provide an anti-plug deployment mechanism to prevent unwanted deployment of the plug.

11. The stent-stabilizing device of claim 8, wherein the aperture diameter is sufficiently small to prevent the grip from entering the hollow bore of the sheath.

12. The stent-stabilizing device of claim 1, wherein the expandable plug member takes the form of an expandable mesh.

13. The stent-stabilizing device of claim 1, wherein the expandable plug member is tapered when in its expanded form to allow easy retraction back into the sheath.

14. A stent-stabilizing device for reducing the relative motion of a stent or other object attached to the distal end of a guide wire with respect to a target lesion in a blood vessel of a patient, comprising:
an elongated sheath, the sheath having proximal and distal opposite open ends and an internal hollow bore disposed between the proximal and distal opposite ends, wherein the diameter of the sheath permits easy ingress into a catheter fitted with a guide wire;
an expandable plug, the plug having first and second opposite ends, wherein the plug comprises a reversibly expandable material, wherein the plug reversibly changes in size between a compressed state and an expanded state such that when the plug is in a compressed state it fits inside the bore of the sheath and when the plug is outside the sheath the plug expands to its expanded state; and
an elongated member having proximal and distal ends, wherein the distal end of the elongated member is connected to the proximal end of the plug, wherein the plug is movable relative to the bore such that the plug is exposed by either sliding the sheath off the plug or by using the elongated member to push the plug out of the sheath to expose the plug thereby causing the plug to expand to its expanded state,
whereby the stent-stabilizing device can be inserted into a guidewire passageway and the expandable plug deployed to hold a section of guide wire steady and thereby reduce any oscillation or movement experienced by the guide wire and a stent or other object attached thereto.

15. The stent-stabilizing device of claim 14, wherein the expandable plug comprises a smart shape memory alloy, further wherein the plug adopts a predetermined first shape and upon compression adopts a predetermined second shape.

16. The stent-stabilizing device of claim 14, wherein the expandable plug a conical shaped Nitinol coiled wire spring that adopts a predetermined first shape and upon compression adopts a predetermined second shape.

17. The stent-stabilizing device of claim 14, wherein the elongated member is a thin stiff wire between about 0.05″ and 0.2″ in diameter.

18. The stent-stabilizing device of claim 14, wherein the elongated member is a thin stiff wire having a diameter of about 0.14″.

19. The stent-stabilizing device of claim 14, wherein the expandable plug comprises Nitinol in the amount of about 55% nickel and 45% titanium.

20. The stent-stabilizing device of claim 14, wherein the expandable plug is constructed of Nitinol coil.

21. The stent-stabilizing device of claim 14, wherein the expandable plug is constructed of Nitinol coil comprising nickel and titanium in the amount of about 55% nickel and 45% titanium.

22. The stent-stabilizing device of claim 14 further comprising a grip attached to the proximal end of the elongated member.

23. The stent-stabilizing device of claim 22, wherein the proximal open end of the sheath defines an aperture of sufficient diameter to accommodate the cross-section area of the elongated member while preventing the expandable plug from inadvertently exiting from the proximal end of the sheath.

24. The stent-stabilizing device of claim 23, wherein the aperture diameter is sufficiently small to prevent the grip from entering the hollow bore of the sheath.
